(19) United States
(12) Patent Application Publication
(10) Pub. No.: US 2024/0424276 A1
(43) Pub. Date: Dec. 26, 2024

(54) WOUND MANAGEMENT ASSEMBLIES, KITS, AND METHODS

(71) Applicant: Reprise Biomedical, Inc., Plymouth, MN (US)

(72) Inventors: Jason Isenburg, Victoria, MN (US); Kyle Bettinger, Blaine, MN (US); Daniel Sorby, Maple Grove, MN (US); Howard Root, Tonka Bay, MN (US)

(73) Assignee: Reprise Biomedical, Inc., Plymouth, MN (US)

(21) Appl. No.: 18/745,650

(22) Filed: Jun. 17, 2024

Related U.S. Application Data
(60) Provisional application No. 63/509,290, filed on Jun. 21, 2023.

(51) Int. Cl. A61M 37/00; A61M 29/00

(52) U.S. Cl. CPC A61M 37/0069; A61M 29/00; A61M 2202/0007; A61M 2202/06; A61M 2202/09

(57) ABSTRACT

Wound management assemblies, kits, and methods are disclosed. A wound management assembly can include a guidewire and a wound matrix compressed onto the guidewire. The wound matrix can optionally be derived from a perfusion-decellularized mammalian tissue or organ. The assembly can further include a tamp member positioned on the guidewire, proximal to a position of the wound matrix. The assembly can be delivered to a desired location within a wound bed using the guidewire or using an introducer set included in a kit. A method can include navigating the wound management assembly to the entrance of the wound bed, using the tamp member to distally urge the wound matrix into the wound bed, and subsequently removing the tamp member, the guidewire, and a sheath if used.

### CLAIM OF PRIORITY

This non-provisional patent document claims the benefit of priority under 35 U.S.C. § 119(e) to U.S. Provisional Patent Application Ser. No. 63/509,290, entitled “WOUND MANAGEMENT ASSEMBLIES, KITS, AND METHODS,” filed on Jun. 21, 2023, which is incorporated herein by reference in its entirety.

### TECHNICAL FIELD

This patent document relates to medical devices. More particularly, but not by way of limitation, the patent document relates to wound management.

### BACKGROUND

Wound management plays a critical role in the healing process of various types of wounds, including partial thickness wounds, full thickness wounds, pressure ulcers, venous ulcers, vascular ulcers, diabetic ulcers, tunneled wounds, undermined wounds, trauma wounds, draining wounds, and surgical wounds.

### OVERVIEW

The present inventors recognize that effective wound management requires the delivery of appropriate wound matrices or scaffolds to a wound bed, which promotes tissue regeneration and healing.

The present subject provides wound management assemblies, kits, and methods for treating wounds in mammals. A wound management assembly comprises a guidewire and a wound matrix compressed onto it. The guidewire does not need to be a metallic wire but must have enough rigidity to allow the wound matrix to be placed onto it, delivered into a wound bed, and then removed after delivery. The wound matrix can optionally be derived from a vascularized portion of a perfusion-decellularized mammalian tissue or organ. The wound matrix can be radially or torsionally compressed onto the guidewire, taking on a variety of cross-sectional shapes, including cylindrical, oval, square, triangle, rectangle, and others. A compressed cylindrical shape is one example. The cylindrical shape may have an initial length of about 3 centimeters (cm) to about 12 cm, inclusive, and an initial diameter of about 2 millimeters (mm) to about 6 mm, inclusive. Post-hydration, the wound matrix can swell from the initial diameter to an expanded diameter that is 1.5 to 5 times the initial diameter.

In one embodiment, the wound management assembly further includes a tamp member positioned on the guidewire, proximal to a position of the wound matrix. The tamp member can be spaced apart from the wound matrix when positioned in a packaging tray to ensure the protection and sterility of the wound matrix. To prevent unintentional sliding of the tamp member off the guidewire, a stopper member can be positioned on the guidewire, proximal to the tamp member.

The wound management assembly can be provided as part of a kit, which includes the wound management assembly and an introducer set comprising a sheath and dilator. The distal tip portion of the introducer set is configured to be placed at the entrance of a wound bed. The sheath provides a conduit for delivering the wound matrix to a desired location within the wound bed.

A method of treating a wound in a mammal can include navigating the wound management assembly, including the guidewire and the wound matrix compressed onto it, to the entrance of a wound bed. Once positioned at or near the entrance of the wound bed, a tamp member can be urged distally against an end of the wound matrix, causing the wound matrix to be pushed off the guidewire into the wound bed. The tamp member and guidewire can then be removed from the mammal, leaving the wound matrix in the wound bed.

These and other examples and features of the present assemblies, kits, and methods will be set forth, at least in part, in the following Detailed Description. This Overview is intended to provide non-limiting examples of the present subject matter—it is not intended to provide an exclusive or exhaustive explanation. The Detailed Description below is included to provide further information about the present assemblies, kits, and methods.

### BRIEF DESCRIPTION OF THE DRAWINGS

In the drawings, like numerals can be used to describe similar features and components throughout the several views. The drawings illustrate generally, by way of example, but not by way of limitation, various assemblies, kits, and methods embodiments discussed in this patent document.

FIG. 1 illustrates an elevation view of a wound management assembly positioned within a packaging tray, as constructed in accordance with at least one embodiment.

FIGS. 2A-2B illustrates an optional introducer set that can be used to deliver a wound management assembly, as constructed in accordance with at least embodiment.

FIGS. 3A-3K illustrate methods of delivering a wound matrix into a wound bed with and without an optional introducer set, as constructed in accordance with at least one embodiment.

FIG. 4 illustrates a flow chart of preparing and applying a wound matrix for treatment of a wound, as constructed in accordance with at least one embodiment.

The drawings are not necessarily to scale. Certain features and components may be exaggerated in scale or in schematic form, and some details may not be shown in the interest of clarity and conciseness.

### DETAILED DESCRIPTION

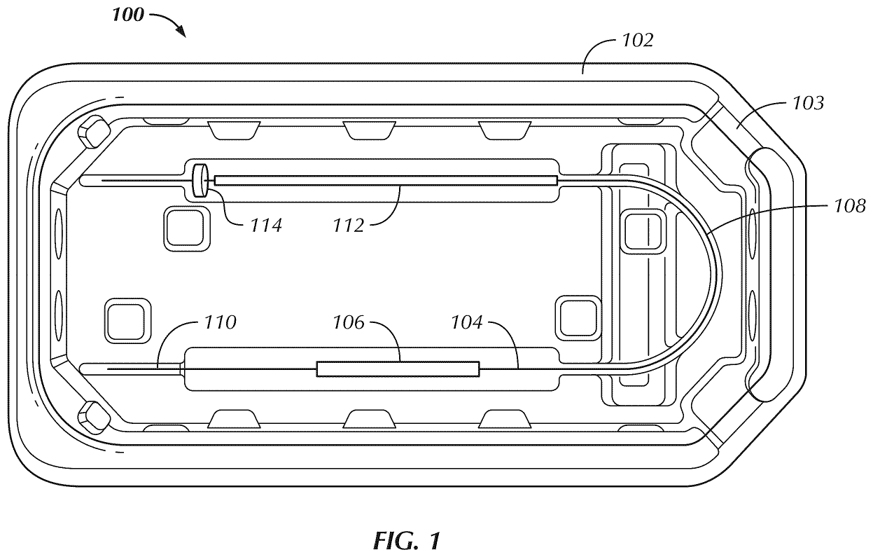

FIG. 1 illustrates an elevation view of a wound management assembly 100 positioned within a packaging tray 102, as constructed in accordance with at least one embodiment. The wound management assembly 100 comprises a guidewire 104 and a wound matrix 106 compressed onto the guidewire. The guidewire 104 can, for example, include a body 108 made of nitinol and a tip 110 made of stainless steel. Nitinol provides flexibility and shape memory to the guidewire 104, allowing it to be easily navigated through a wound bed. The stainless-steel tip 110 provides strength and durability to the guidewire 104, facilitating its insertion and maneuverability.

The wound matrix 106 can be derived from a vascularized portion of a perfusion-decellularized mammalian tissue or organ. By way of example, this tissue or organ can be a perfusion-decellularized porcine liver. The wound matrix 106 serves as a scaffold for tissue regeneration and healing when placed within the wound bed. The perfusion-decellularization process ensures the removal of cellular components while preserving the extracellular matrix structure, providing a suitable scaffold for tissue regeneration and healing.

To achieve decellularization, the techniques disclosed in U.S. Pat. No. 8,470,520, titled “Decellularization and Recellularization of Organs and Tissues,” issued on Jun. 25, 2013, to Ott et al., which is incorporated herein by reference in its entirety, can be employed. This patent describes methods and materials to decellularize a mammalian organ or tissue, including cannulating the organ or tissue and perfusing the cannulated organ or tissue with a cellular disruption medium. Upon completion of the perfusion-decellularization process, an acellular collagen scaffold is obtained.

The decellularized material can be aerated while preserving its structure, as disclosed in U.S. Pat. No. 9,974,814, titled “Gas Filled Decellularized Matrix,” issued on May 22, 2018, to Katane et al., and as disclosed in commonly owned U.S. patent application Ser. No. 17/834,561, titled “Dry Inflated Decellularized Extracellular Matrix,” filed on Jun. 7, 2022, by Isenburg, each of which is incorporated herein by reference in its entirety. Together, these patent matters describe methods to provide an inflated and suspension-dried decellularized extracellular matrix of a mammalian organ or tissue, or a vascularized portion thereof.

The wound matrix 106 is compressed onto the guidewire 104 in a radial or torsional manner, resulting in a compressed shape. One example is a cylindrical shape, which can facilitate insertion into the wound bed and ensures proper positioning and stability. However, the compressed wound matrix can take on various cross-sectional shapes, including oval, square, triangle, rectangle, and others. By way of example, the wound matrix's 106 cylindrical shape may have an initial length of about 3 cm to about 12 cm, inclusive, and an initial diameter of about 2 mm to about 6 mm, inclusive. Expanded dimensions, both larger and smaller, are also contemplated by the inventors. These dimensions can vary depending on the size and type of wound being treated. In varying examples, the wound matrix 106 starts as an uncompressed, rectangular segment of matrix material.

In one embodiment, the wound management assembly further comprises a tamp member 112 positioned over the guidewire 104, proximal to a position of the wound matrix 106. The tamp member 112 provides a means for distally urging or pushing the wound matrix 106 off the guidewire 104 and into the wound bed during the treatment process. The tamp member 112 can vary in cross-sectional size, such as diameter, to correspond to the cross-sectional size of the compressed wound matrix 106. It can be a tamp tube made of biocompatible Arkema PEBAX 7033 SA01 medical-grade material. To prevent unintentional sliding of the tamp member 112 off the guidewire 104 during the removal of the wound management assembly from a packaging tray 102, a stopper member 114 can be positioned on the guidewire, proximal to the tamp member 112. The stopper member 114 can be a silicone foam stopper disc, for example.

In varying embodiments, the wound matrix 106 is provided in a dry state. This dry state allows for ease of handling, storage, and transportation. The wound matrix 106 can be hydrated within the wound bed, either through natural wound exudate or by introducing a suitable fluid. Upon hydration within the wound bed, the wound matrix 106 can swell from its initial diameter to an expanded diameter that is 1.5 to 5 times the initial diameter. This expansion promotes contact between the wound matrix 106 and the wound bed, facilitating tissue regeneration and healing.

The wound management assembly 100 is a wound management product, and certain packaging materials, components, and sterilization methods can be employed for its safe storage and delivery. The wound management assembly 100 can be packaged in a molded tray 102 (e.g., a polyethylene terephthalate glycol (PETG) tray) with a snap-on lid 103 made of the same material, as depicted in FIG. 1. The tamp member 112 and the stopper member 114 can be positioned in such a way that they do not come into direct contact with the wound matrix 106 within the tray 102 to ensure the protection and sterility of the wound matrix 106 during storage and transportation. The tray 102 and lid 103 can be sealed in a laminated aluminum pouch. This pouch can undergo sterilization using e-beam irradiation, for example.

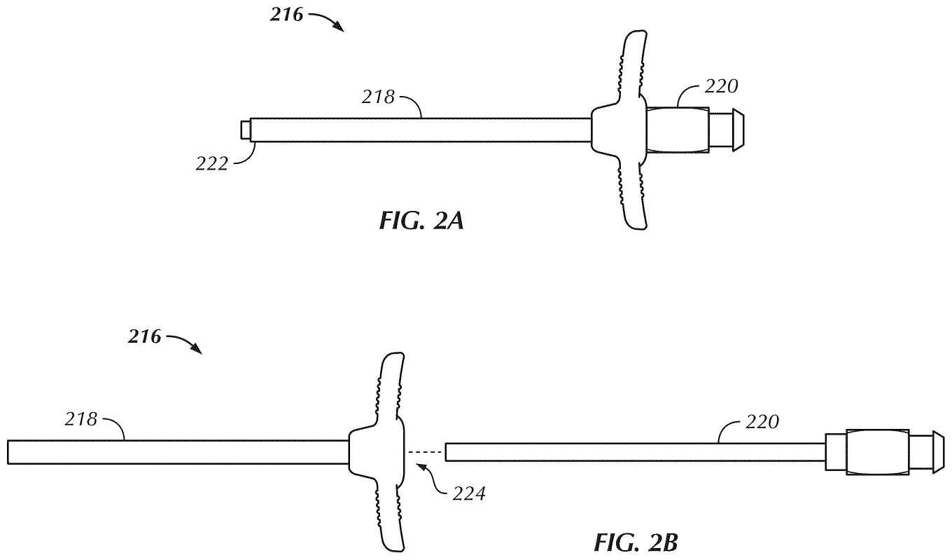

FIGS. 2A-B illustrate an optional introducer set 216 that can be used to deliver a wound management assembly, as constructed in accordance with at least embodiment. The wound management assembly can be provided as part of a kit, which further comprises the introducer set 216, including a sheath 218 and dilator 220. The sheath 218 can be, but need not be, a tearaway sheath. The sheath 218 and dilator 220 can be made of polypropylene and high-density polyethylene (HDPE), respectively, and can be sized based on the diameter of a wound matrix. For example, a 10 French (F) sheath can correspond to a wound matrix having a diameter of 3 mm, and a 16F sheath can correspond to a wound matrix having a diameter of 5 mm.

The sheath 218 and dilator 220 can be used to assist in delivering the wound matrix to the desired location within a wound bed. The distal tip 222 portion of the introducer set 216 is configured to be placed at or within the entrance of the wound bed, allowing for precise placement and controlled delivery of the wound matrix. The sheath 218 provides a conduit 224 through which the wound matrix can be delivered to the desired location within the wound bed, and its construction allows for easy removal after the wound matrix has been deployed through it.

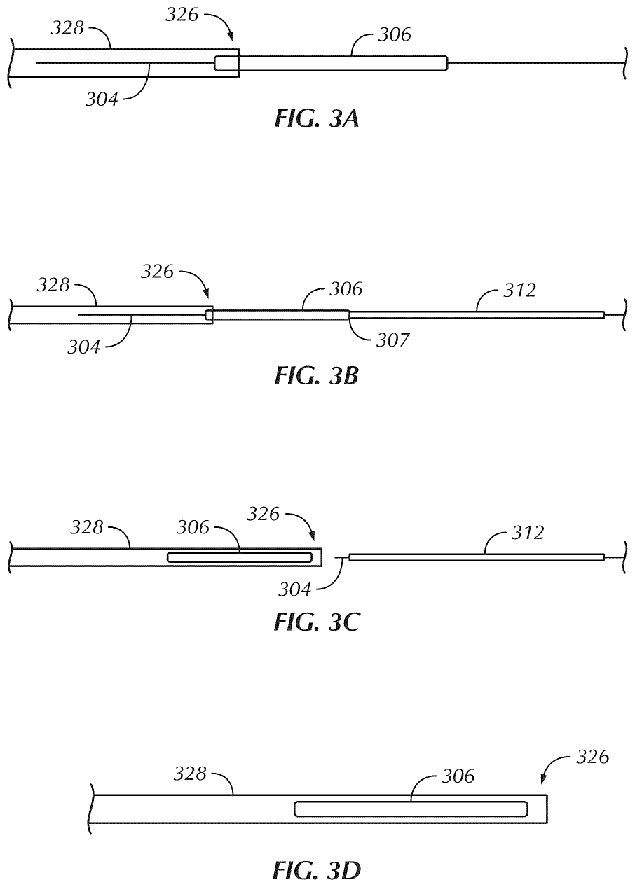

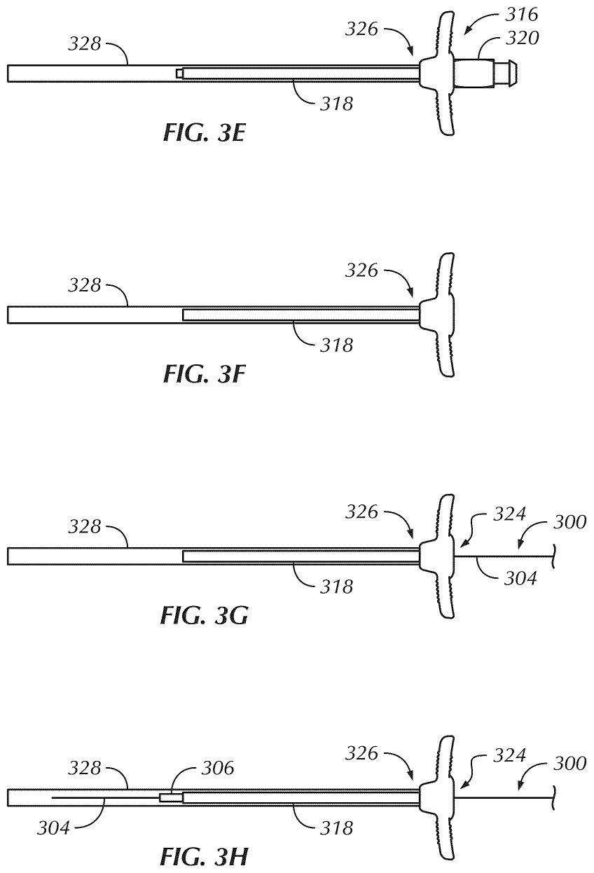

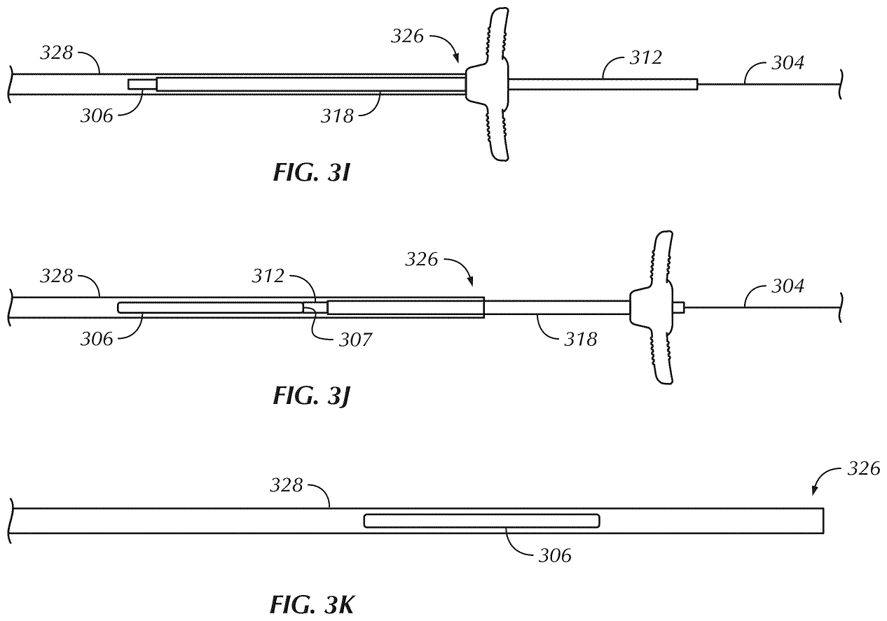

FIGS. 3A-3K illustrate methods of delivering a wound matrix into a wound bed with and without an optional introducer set, as constructed in accordance with at least one embodiment. The method of treating a wound in a mammal using the wound management assembly can involve several steps. First, the wound management assembly, including a guidewire and the wound matrix compressed onto it, is navigated to the entrance of the wound bed.

There are two approaches to navigate to the wound area 326: using the guidewire 304, as shown in FIGS. 3A-3D, or through the sheath 318, as shown in FIGS. 3E-3K. When using the guidewire 304, the clinician first positions themselves near the wound area 326 and inserts the guidewire 304, as shown in FIG. 3A. Once properly positioned, the clinician distally slides a tamp member 312 along the guidewire 304 to a proximal end 307 of the wound matrix 306, as shown in FIG. 3B. The clinician then pushes the wound matrix 306 off the guidewire 304 and into the wound bed 328, as shown in FIG. 3C. After successful placement, the guidewire 304 and tamp member 312 are removed, leaving the wound matrix 306 in the wound bed 328, as shown in FIG. 3D.

Alternatively, if the sheath 318 is used, the introducer set 316-including the sheath 318 and dilator 320—is positioned in the wound bed 328, as shown in FIG. 3E. The dilator 320 is then removed, as shown in FIG. 3F, and the wound management assembly 300 is deployed through a conduit 324 of the in-place sheath 318, as shown in FIGS. 3G and 3H. Once the wound management assembly 300 is positioned at or near the entrance of the wound bed 328, a tamp member 312 is urged distally against a proximal end of the wound matrix 306, as shown in FIG. 31. This urging or pushing action against the proximal end 307 of the wound matrix 306 causes the matrix to be pushed off the guidewire 304 and into the wound bed 328, as shown in FIG. 3J. Subsequently, the sheath 318 is removed along with the guidewire 304 and tamp member 312, leaving the wound matrix 306 in the wound bed 328, as shown in FIG. 3K.

In an alternative method (not shown), clinicians may manually remove the wound matrix 306 from the guidewire 304 and place it into the wound bed 328 using general surgical tools or their hands.

In varying embodiments, the wound matrix 306 is dry and compressed when slid off the guidewire 304.

Once deployed in the wound bed 328, the wound matrix 306 is designed to become hydrated by wound fluids. Alternatively, clinicians may choose to hydrate the wound matrix 306 with a sterile saline or Lactated Ringers solution flush in situ. Upon hydration, the wound matrix 306 will relax and expand to conform to the shape of the wound bed 328, maximizing contact with the wound bed and facilitating tissue regeneration and healing.

The wound matrix 306 remains in the wound bed 328, providing a scaffold for tissue regeneration and healing. The method may involve allowing sufficient time for the wound matrix 306 to be resorbed within the wound bed 328. The resorption process ensures that the wound matrix 306 is gradually replaced by new tissue, leading to the healing of the wound. The method of treating a wound according to the present subject matter can be applied to various types of wounds, including partial thickness wounds, full thickness wounds, pressure ulcers, venous ulcers, vascular ulcers, diabetic ulcers, tunneled wounds, undermined wounds, trauma wounds (e.g., abrasions, lacerations, partial-thickness burns, and skin tears), draining wounds, and surgical wounds (e.g., donor sites/grafts, post-Mohs surgery, post-laser surgery, podiatric, and wound dehiscence).

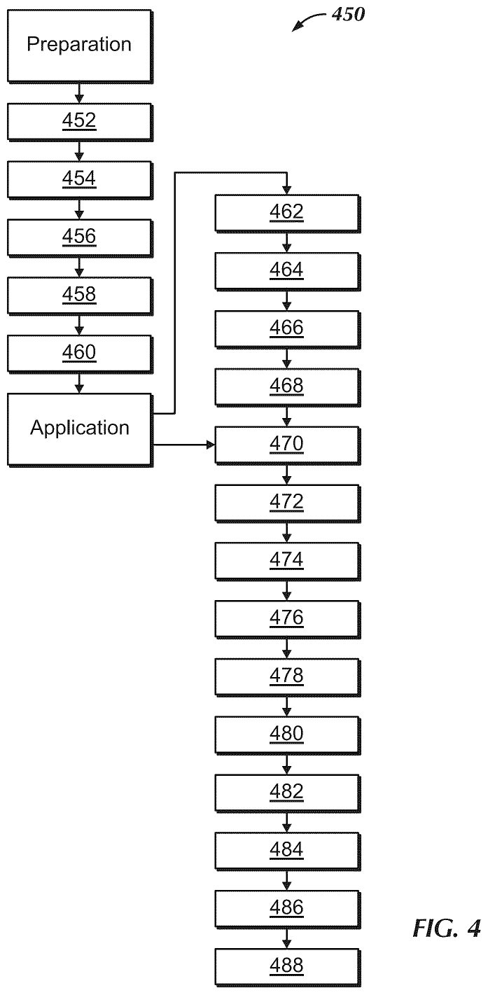

FIG. 4 illustrates a flow chart 450 of preparing and applying a wound matrix for treatment of a wound, as constructed in accordance with at least one embodiment.

To prepare a wound matrix for application, the following steps can be followed:
1. At 452, open a carton and remove pouches containing a wound management assembly, including a guidewire, a wound matrix, a tamp member and a stopper member, and an introducer set, including a sheath and a dilator.
2. At 454, inspect the pouches and ensure they are undamaged with intact seals.
3. At 456, open the pouches aseptically and transfer a tray including the wound management assembly and the introducer set into a sterile field.
4. At 458, once within the sterile field, remove the plastic lid from the wound management assembly tray.
5. At 460, after removing the wound management assembly from the tray, discard the stopper member located at the distal portion of the guidewire. The stopper member is for packaging purposes and ensures the tamp member remains securely attached to the guidewire during shipment.

To apply the wound matrix, the wound should be prepared using standard clinical practice methods, ensuring the removal of debris and devitalized tissue. Initial debridement may be necessary to ensure the wound's edges contain viable tissue. If using the optional introducer set, the following steps can be followed. Otherwise, the clinician can proceed directly to step 470.
1. At 462, place the introducer set in the desired location within the wound.
2. At 464, once the sheath is positioned correctly, remove the dilator.
3. At 466, locate the guidewire and wound matrix and remove and discard the stopper member from the distal end of the tamp member.
4. At 468, insert the guidewire with the wound matrix and tamp member through a conduit of the sheath.
5. At 470, ensure the removal of the stopper member from the guidewire's end. Then, using aseptic techniques, guide the tip of the guidewire to the desired terminal end of the wound.
6. At 472, distally slide the tamp member to an end of the wound matrix and gently push the wound matrix off the guidewire into the wound. The wound matrix should be placed in maximum possible contact with healthy, well-vascularized tissue, acting as a scaffold to provide a protective environment for the wound.
7. At 474, if the sheath was used is a tearaway sheath, grasp the butterfly hub, and separate it. Carefully peel the tearaway sheath down, exposing the tamp tube, guidewire, and the placed wound matrix. Alternatively, if not using the tearaway function, the sheath can be removed as a whole. Remove the remaining sheath, tamp member, and guidewire, leaving the wound matrix in place.
8. At 476, using sterile scissors or scalpel, carefully trim any excess wound matrix outside of the wound space.
9. At 478, flush the wound containing the wound matrix with sterile saline or Lactated Ringer's solution to allow hydration, relaxation, and conformation of the matrix to the wound bed. Alternatively, the wound bed itself could naturally hydrate the wound matrix without the use of sterile saline or Lactated Ringer's solution.
10. At 480, apply an appropriate non-adherent primary wound dressing over the wound matrix to maintain matrix adherence and protect the wound area.
11. At 482, apply a suitable secondary dressing that can manage wound exudate, keep the wound matrix moist, and securely hold it in place.
12. At 484, dispose of the guidewire, tamp member, stopper member, sheath and dilator, and packaging in compliance with accepted medical practices.
13. At 486, during the healing process, some sections of the wound matrix may not integrate. If necessary, carefully remove any loose product. However, do not remove any integrated portions of the wound matrix.
14. At 488, if the wound is free of infection and necrosis but not fully epithelialized, follow standard clinical protocol for additional application or therapy.

### Closing Notes:

The wound management assemblies, kits, and methods described herein provide an effective and controlled approach to wound management. The compressed wound matrix, which can optionally be derived from a perfusion-decellularized mammalian tissue or organ, allows for precise delivery and promotes tissue regeneration and healing within the wound bed, especially in tunneled or undermined wound beds. The kit components and method steps ensure ease of use and accurate placement of the wound matrix, leading to improved wound treatment outcomes.

The above Detailed Description includes references to the accompanying drawings, which form a part of the Detailed Description. The Detailed Description should be read with reference to the drawings. The drawings show, by way of illustration, specific embodiments in which the present assemblies, kits, and methods can be practiced. These embodiments are also referred to herein as “examples.”

The above Detailed Description is intended to be illustrative and not restrictive. For example, the above-described examples (or one or more features or components thereof) can be used in combination with each other. Also, various features or components have been or can be grouped together to streamline the disclosure. This should not be interpreted as intending that an unclaimed disclosed feature is essential to any claim. Rather, inventive subject matter can lie in less than all features of a particular disclosed embodiment. Thus, the following claim examples are hereby incorporated into the Detailed Description, with each example standing on its own as a separate embodiment.

In Example 1, a wound management assembly can comprise a guidewire and a wound matrix compressed onto the guidewire. The wound matrix can be derived from a vascularized portion of a perfusion-decellularized mammalian tissue or organ.

In Example 2, the wound management assembly of Example 1 can optionally further comprise a tamp member. The tamp member can be a tamp tube positioned over the guidewire, proximal to a position of the wound matrix.

In Example 3, the wound management assembly of Example 2 can optionally be configured such that the tamp member is spaced apart from the wound matrix when positioned in a packaging tray.

In Example 4, the wound management assembly of any one of Examples 2 or 3 can optionally further comprise a stopper member positioned on the guidewire, proximal to the tamp member. The stopper member is configured to inhibit the tamp member from unintentionally sliding off the guidewire.

In Example 5, the wound management assembly of any one or any combination of Examples 1-4 can optionally be configured such that the wound matrix is radially compressed onto the guidewire.

In Example 6, the wound management assembly of any one or any combination of Examples 1-4 can optionally be configured such that the wound matrix is torsionally compressed onto the guidewire.

In Example 7, the wound management assembly of any one or any combination of Examples 1-6 is optionally configured such that the wound matrix has a compressed cylindrical shape.

In Example 8, the wound management assembly of Example 7 is optionally configured such that the cylindrical shape has an initial length of about 3 cm to about 12 cm, inclusive.

In Example 9, the wound management assembly of any one of Examples 7 or 8 is optionally configured such that the cylindrical shape has an initial diameter of about 2 mm to about 6 mm, inclusive.

In Example 10, the wound management assembly of Example 9 is optionally configured such that the wound matrix swells from the initial diameter to an expanded diameter that is 1.5 to 5 times the initial diameter, post-hydration.

In Example 11, the wound management assembly of any one or any combination of Examples 1-10 is optionally configured such that the wound matrix is dry and aerated.

In Example 12, the wound management assembly of any one or any combination of Examples 1-11 is optionally configured such that the perfusion-decellularized mammalian tissue or organ is a perfusion-decellularized porcine liver.

In Example 13, the wound management assembly of any one or any combination of Examples 1-12 is optionally configured such that the guidewire includes a body including nitinol and a tip including stainless steel.

In Example 14, a kit comprises the wound management assembly of any one or any combination of Examples 1-13, and an introducer set including a sheath and a dilator.

In Example 15, the kit of Example 14 is optionally configured such that a distal tip portion of the introducer set is configured to be placed at an entrance of a wound bed, and the sheath provides a conduit for delivering the wound matrix to a desired location within the wound bed.

In Example 16, a method of treating a wound in a mammal comprises navigating a wound management assembly to an entrance of a wound bed. The wound management assembly includes a guidewire and a wound matrix compressed onto the guidewire. Once the wound management assembly is positioned at or near the entrance of the wound bed, a tamp member is employed to exert pressure against an end of the wound matrix. This pressure facilitates the detachment of the wound matrix from the guidewire, allowing it to be pushed into the wound bed. After the wound matrix is successfully placed in the wound bed, the tamp member and the guidewire are carefully removed, leaving the wound matrix in the wound bed.

In Example 17, the method of Example 16 is optionally configured such that the wound matrix is derived from a vascularized portion of a perfusion-decellularized mammalian tissue or organ.

In Example 18, the method of any one of Examples 16 or 17 is optionally configured such that navigating the wound management assembly to the entrance of the wound bed includes positioning a distal tip portion of the guidewire in the wound bed.

In Example 19, the method of any one or any combination of Examples 16-18 can optionally be configured such that navigating the wound management assembly to the entrance of the wound bed includes positioning a distal tip portion of an introducer set, including a sheath and dilator, in the wound bed.

In Example 20, the method of Example 19 optionally further comprises removing the dilator from the sheath, and then advancing the wound management assembly through a conduit of the sheath.

In Example 21, the method of any one or any combination of Examples 16-20 can optionally be configured such that urging the tamp member against the end of the wound matrix includes sliding the tamp member in a distal direction along the guidewire.

In Example 22, the method of Example 21 is optionally configured such that the wound matrix is a dry, compressed matrix when slid off the guidewire into the wound bed.

In Example 23, the method of Example 22 optionally further comprises hydrating the dry, compressed matrix in the wound bed, including expanding the matrix from an initial diameter to an expanded diameter that is 1.5 to 5 times the initial diameter.

In Example 24, the method of any one or any combination of Examples 16-23 optionally further comprises allowing sufficient time for the wound matrix to be resorbed within the wound bed.

In Example 25, the method of any one or any combination of Examples 16-24 is optionally configured such that treating the wound includes treating a partial thickness wound, a full thickness wound, a pressure ulcer, a venous ulcer, a vascular ulcer, a diabetic ulcer, a tunneled wound, an undermined wound, a trauma wound, a draining wound, or a surgical wound.

In Example 26, the wound management assembly, kit, or method of any one or any combination of Examples 1-25 can optionally be configured such that all components or options recited are available to use or select from.

Certain terms are used throughout this patent document to refer to features or components. As one skilled in the art will appreciate, different people may refer to the same feature or component by different names. This patent document does not intend to distinguish between components or features that differ in name but not in function.

For the following defined terms, certain definitions shall be applied unless a different definition is given elsewhere in this patent document. The terms “a,” “an,” and “the” are used to include one or more than one, independent of any other instances or usages of “at least one” or “one or more.” The term “or” is used to refer to a nonexclusive or, such that “A or B” includes “A but not B,” “B but not A,” and “A and B.” All numeric values are assumed to be modified by the term “about,” whether explicitly indicated. The term “about” refers to a number or a range of numbers that one skilled in the art considers equivalent to the recited value (e.g., having the same function or result). The recitation of numerical ranges by endpoints includes all numbers and sub-ranges within and bounding that range (e.g., 1 to 4 includes 1, 1.5, 1.75, 2, 2.3, 2.6, 2.9, etc. and 1 to 1.5, 1 to 2, 1 to 3, 2 to 3.5, 2 to 4, 3 to 4, etc.). The terms “distal” and “proximal” are used to refer to a position or direction relative to an operating clinician. “Distal” and “distally” refer to a position that is distant from, or in a direction away from, the clinician. “Proximal” and “proximally” refer to a position that is near, or in a direction toward, the clinician. The term “wound matrix compressed onto the guidewire” or similar includes both a wound matrix that is directed compressed onto the guidewire and a wound matrix that is first compressed and then placed (e.g., threaded) onto the guidewire.

The scope of the present assemblies, kits, and methods should be determined with reference to the appended claims, along with the full scope of equivalents to which such claims are entitled. In the appended claims, the terms “including” and “in which” are used as the plain-English equivalents of the respective terms “comprising” and “wherein.” Also, in the following claims, the terms “including” and “comprising” are open-ended; that is, an assembly, kit, or method that includes features or components in addition to those listed after such a term in a claim are still deemed to fall within the scope of that claim. Moreover, in the following claims, the terms “first,” “second,” and “third,” etc. are used merely as labels and are not intended to impose numerical requirements on their objects.

The Abstract is provided to allow the reader to quickly ascertain the nature of the technical disclosure. It is submitted with the understanding that it will not be used to interpret or limit the scope or meaning of the claims.

1. A wound management assembly, comprising:
a guidewire; and
a wound matrix in a dry, unexpanded state radially compressed over the guidewire,
wherein the wound matrix is porous in all directions and configured to expand when hydrated within a wound bed, either through natural wound exudate or by introducing a fluid.

2. The wound management assembly of claim 1, further comprising a tamp member positioned on the guidewire, proximal to a position of the wound matrix.

3. The wound management assembly of claim 2, further comprising a stopper member positioned on the guidewire, proximal to the tamp member, wherein the stopper member is configured to inhibit the tamp member from unintentionally sliding off a proximal end of the guidewire.

4. (canceled)

5. The wound management assembly of claim 1, wherein the wound matrix has a compressed cylindrical shape.

6. The wound management assembly of claim 5, wherein the cylindrical shape has an initial diameter of about 2 millimeters (mm) to about 6 mm, inclusive.

7. The wound management assembly of claim 6, wherein the wound matrix swells from the initial diameter to an expanded diameter that is 1.5 to 5 times larger than the initial diameter, post-hydration.

8. The wound management assembly of claim 1, wherein the wound matrix is dried through suspension aeration.

9. The wound management assembly of claim 1, wherein the wound matrix is derived from a vascularized portion of a perfusion-decellularized mammalian tissue or organ.

10. A kit, comprising:
the wound management assembly of claim 1; and
an introducer set including a sheath and a dilator.

11. The kit of claim 10, wherein a distal tip portion of the introducer set is configured to be placed at an entrance of a wound bed, and wherein the sheath provides a conduit for delivering the wound matrix to a desired location within the wound bed.

12. A method of treating a wound in a mammal, comprising:
performing an initial debridement to ensure a wound bed's edges contain viable tissue and promote blood flow;
navigating a wound management assembly, which includes a guidewire and a wound matrix in a dry, unexpanded state radially compressed over the guidewire, to an entrance of the wound bed;
once positioned at or near the entrance of the wound bed, urging a tamp member against an end of the wound matrix, thereby pushing the wound matrix off the guidewire and into the wound bed;
removing the tamp member and the guidewire from the mammal, leaving the wound matrix to expand within the wound bed when hydrated through the absorption of natural wound exudate or introduced fluid into its pores; and
ensuring no sealing compound is delivered into the wound bed above or around the wound matrix, such that hemostasis is not the objective, but rather blood flow is encouraged.

13. The method of claim 12, wherein the wound matrix is derived from a vascularized portion of a perfusion-decellularized mammalian tissue or organ.

14. The method of claim 12, wherein navigating the wound management assembly to the entrance of the wound bed includes positioning a distal tip of the guidewire in the wound bed.

15. The method of claim 12, wherein navigating the wound management assembly to the entrance of the wound bed includes positioning a distal tip portion of an introducer set, which includes a sheath and dilator, in the wound bed.

16. The method of claim 15, further comprising removing the dilator from the sheath, and then advancing the wound management assembly through a conduit of the sheath.

17. The method of claim 12, wherein urging the tamp member against the end of the wound matrix includes sliding the tamp member in a distal direction along the guidewire.

18. The method of claim 17, wherein the wound matrix is configured to expand within the wound bed from an initial diameter to an expanded diameter that is 1.5 to 5 times larger than the initial diameter.

19. The method of claim 12, further comprising allowing sufficient time for the wound matrix to be resorbed within the wound bed.

20. The method of claim 12, wherein treating the wound includes treating a partial thickness wound, a full thickness wound, a pressure ulcer, a venous ulcer, a vascular ulcer, a diabetic ulcer, a tunneled wound, an undermined wound, a trauma wound, a draining wound, or a surgical wound.
